(19) United States
(12) Patent Application Publication
(10) Pub. No.: US 2020/0381126 A1
(43) Pub. Date: Dec. 3, 2020

(54) DIAGNOSTIC PROBABILITY CALCULATOR

(71) Applicant: Pearson Education, Inc., Bloomington, MN (US)

(72) Inventor: Kristina BREAUX, Houston, TX (US)

(21) Appl. No.: 16/429,945

(22) Filed: Jun. 3, 2019

(51) Int. Cl. G16H 50/30; G16H 10/20; G06N 7/00; G06F 17/18; G06F 3/0482

(52) U.S. Cl. CPC G16H 50/30; G16H 10/20; G06F 3/0482; G06F 17/18; G06N 7/005

(57) ABSTRACT

Systems and methods of the present invention provide for one or more server computers communicatively coupled to a network and configured to: receive, from a GUI on a user device, user input including a determination of whether a prior probability of dyslexia exists for a user, a selection of a dyslexia screening test administered to the user and an indication of whether the test indicated a risk of dyslexia, and if so, calculate a Bayesian positive predictive value. If not, the system calculates a Bayesian negative predictive value. The system then generates a report GUI including the Bayesian positive or negative predictive value, a probability of the user having dyslexia, and a recommendation, according to the probability of the user having dyslexia, representing an intensity of a treatment evaluation response.

### FIELD OF THE INVENTION

This disclosure relates to the field of systems and methods configured to use screening tests or other diagnostic evaluation processes that incorporate known risk factors and assessment data, to objectively quantify a student's risk level for dyslexia or other conditions.

### SUMMARY OF THE INVENTION

The present invention provides systems and methods comprising one or more server hardware computing devices or client hardware computing devices, communicatively coupled to a network, and each comprising at least one processor executing specific computer-executable instructions within a memory that, when executed, cause the system to: receive, from a GUI on a user device, user input including a determination of whether a prior probability of dyslexia exists for a user, a selection of a dyslexia screening test administered to the user and an indication of whether the test indicated a risk of dyslexia, and if so, calculate a Bayesian positive predictive value. If not, the system calculates a Bayesian negative predictive value. The system then generates a report GUI including the Bayesian positive or negative predictive value, a probability of the user having dyslexia, and a recommendation, according to the probability of the user having dyslexia, representing an intensity of a treatment evaluation response.

The above features and advantages of the present invention will be better understood from the following detailed description taken in conjunction with the accompanying drawings.

### BRIEF DESCRIPTION OF THE DRAWINGS

FIG. 1 illustrates a system level block diagram for a network-based system for quantifying a risk of a specific condition based on screening tests and other diagnostic evaluation processes.

FIG. 2 illustrates a system level block diagram for a network-based system for quantifying a risk of a specific condition based on screening tests and other diagnostic evaluation processes.

FIG. 3 illustrates a non-limiting example embodiment including a Graphical User Interface (GUI) used for receiving user input used to determine if a user has a prior probability of a specific condition.

FIG. 4 illustrates a non-limiting example embodiment including a GUI used for receiving user input used to identify a first screening test administered to the user, and determining if the screening test indicates a risk of a specific condition.

FIG. 5 illustrates a non-limiting example embodiment including a GUI used for receiving user input used to determine if a second screening test was administered to a user, and identify the second screening test.

FIG. 6 illustrates a non-limiting example embodiment including a GUI used for receiving user input used to determine if the second screening test indicates a risk of a specific condition.

FIG. 7 illustrates a non-limiting example embodiment including a GUI used for displaying a generated report indicating a risk of a specific condition.

FIG. 8 illustrates a flow diagram including non-limiting example method steps for calculating a risk of a specific condition.

### DETAILED DESCRIPTION

The present inventions will now be discussed in detail with regard to the attached drawing figures that were briefly described above. In the following description, numerous specific details are set forth illustrating the Applicant's best mode for practicing the invention and enabling one of ordinary skill in the art to make and use the invention. It will be obvious, however, to one skilled in the art that the present invention may be practiced without many of these specific details. In other instances, well-known machines, structures, and method steps have not been described in particular detail in order to avoid unnecessarily obscuring the present invention. Unless otherwise indicated, like parts and method steps are referred to with like reference numerals.

Bayesian statistics have been used in evidence based sciences to help practitioners provide better, more accurate analysis and recommendations, thereby making the analysis of scientific data and results more efficient. Currently, there is a groundswell of interest and political pressure to understand and identify dyslexia in the learning process. Dyslexia researchers have also begun to calculate the probability of dyslexia, but it's new to educators and practitioners in the field, and Bayesian statistics and dyslexia clinical assessments have not been applied to this area of research.

Despite the current groundswell of interest in understanding and identifying dyslexia, professional training is lacking, there is widespread under-identification of dyslexia, and the subjects who are identified are not always given the right amount of intervention. One additional problem is that practitioners know that family history of dyslexia is important to consider (a person is 4-5 times more likely to have the condition if a first degree relative is affected), but there is currently no way to quantity family history information in the context of screening or evaluation. Thus, the education market, and other learning organizations, would benefit from a tool that incorporates Bayesian statistics.

In the disclosed embodiments, a probability calculator system may use screening tests or other diagnostic evaluation processes that incorporate known risk factors and assessment data, to objectively quantify a student's risk level for dyslexia. The probability calculator system may use this data and the results to execute calculations which provide a report including both the level of risk or probability of dyslexia for a subject or other user, and recommendations for an amount of intervention needed based on the level of risk. The calculator may be scaled up to more efficiently compute the risk factors for large groups of students.

The calculation incorporates family history for dyslexia, the prevalence rate of dyslexia, the results of one or more dyslexia screening tests, and the psychometric properties of the administered screening test, or in other words, the accuracy with which it classifies subjects or other users as having or not having dyslexia.

The option to use multiple screening tests is best practice for a time efficient workflow that minimizes false positives. The type of probability calculation (Bayes theorem) enables professionals to quantify a person's risk for a condition, which informs their diagnosis. The level of severity (probability) can inform the intensity of the treatment evaluation response. The resulting probabilities are interpreted categorically (low, medium, high) to provide a triage mechanism for tailoring recommendations to the degree of risk severity.

As described in more detail herein, the probability calculator system may include various computing device and software components, including an electronic data store coupled to a network, storing data related to the one or more screening tests and the sensitivity, specificity, false positive data, and false negative data in association with each of the screening tests.

The probability calculator system may further include one or more servers. Each of these servers may be a computing device coupled to a network and including at least one processor that executes instructions within a memory. For example, the probability calculator system may include one or more probability calculator software modules executing in the server(s)' memory to execute the method steps disclosed herein.

For example, these software modules may include instructions so that one or more servers are configured to generate a GUI for display on one or more client devices that are also included within the probability calculator system. This GUI may include requests for user input, and a GUI control for each of the user input requests. Specifically, a first request and GUI control may request, and receive user input from a user operating the client device, input indicating whether a prior probability of dyslexia exists for the user. A second request and GUI control may request, and receive user input selecting a dyslexia screening test administered to the user. A third request and GUI control may request, and receive user input indicating whether the dyslexia screening test indicated a risk of dyslexia. The client device may then transmit the first, second, and third user input through the network to the one or more servers.

The server may receive the first, second, and third user inputs from the client device, as input into the GUI, and in response, automatically determine whether the third GUI control input indicated whether the dyslexia screening test indicated a risk of dyslexia for the user. If so, the server may automatically access the data store, to identify the sensitivity, specificity, false positive data, and false negative data stored in association with the screening test selected by the second GUI control input. Using the sensitivity and specificity data, the server may automatically calculate a Bayesian positive predictive value.

However, if the third GUI control input indicates that the dyslexia screening test did not indicate a risk of dyslexia, the server may automatically use the sensitivity and specificity data stored in association with the screening test selected by the second GUI control input to automatically calculate a Bayesian negative predictive value.

The electronic data store may further store a framework associated with results of the calculation of the Bayesian positive predictive value or the Bayesian negative predictive value. This framework may define various levels of risk for dyslexia, according to the predictive values. The framework may further include recommendations associated with the various levels defined in the framework within the data store.

After calculating the Bayesian positive predictive value or the Bayesian negative predictive value, the server may access the framework stored within the data store, and automatically select the identified level and the recommendations associated in the data store with the identified level, according to the predictive values calculated.

The server may then automatically generate a report GUI, including the Bayesian positive or negative predictive value, a probability, based on the predictive values of whether the user has dyslexia, and a recommendation, according to the probability of the user having dyslexia and the selected data from the framework, representing an intensity of a treatment evaluation response. Once generated, the server may automatically transmit the report GUI through the network for display on the client device.

The disclosed embodiments offer the following advantages: First, as a triage mechanism, the disclosed embodiments allow practitioners to quickly determine which individuals are at the highest risk, or highest severity, for dyslexia or other conditions, warranting an aggressive evaluation/treatment response. Next, the disclosed embodiments provide a level of decision support that encourages customers to use screening tests (e.g., Pearson dyslexia tests) and stay within a provider's ecosystem. Finally, it improves the predictive validity of tests to help customers make better decisions for students. The probability calculator system is designed to help learners at highest risk get the best resources and the most intensive interventions sooner.

These example embodiments are non-limiting. In addition to dyslexia, the probability calculator system may easily be applied to other diagnoses, and to additional products in other areas/markets.

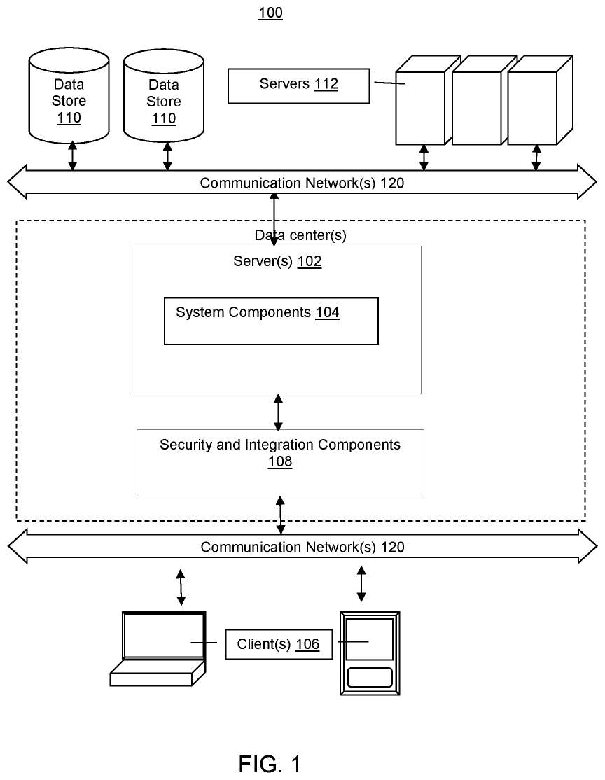

FIG. 1 illustrates a non-limiting example distributed computing environment 100, which includes one or more computer server computing devices 102, one or more client computing devices 106, and other components that may implement certain embodiments and features described herein. Other devices, such as specialized sensor devices, etc., may interact with client 106 and/or server 102. The server 102, client 106, or any other devices may be configured to implement a client-server model or any other distributed computing architecture.

Server 102, client 106, and any other disclosed devices may be communicatively coupled via one or more communication networks 120. Communication network 120 may be any type of network known in the art supporting data communications. As non-limiting examples, network 120 may be a local area network (LAN; e.g., Ethernet, Token-Ring, etc.), a wide-area network (e.g., the Internet), an infrared or wireless network, a public switched telephone networks (PSTNs), a virtual network, etc. Network 120 may use any available protocols, such as (e.g., transmission control protocol/Internet protocol (TCP/IP), systems network architecture (SNA), Internet packet exchange (IPX), Secure Sockets Layer (SSL), Transport Layer Security (TLS), Hypertext Transfer Protocol (HTTP), Secure Hypertext Transfer Protocol (HTTPS), Institute of Electrical and Electronics (IEEE) 802.11 protocol suite or other wireless protocols, and the like.

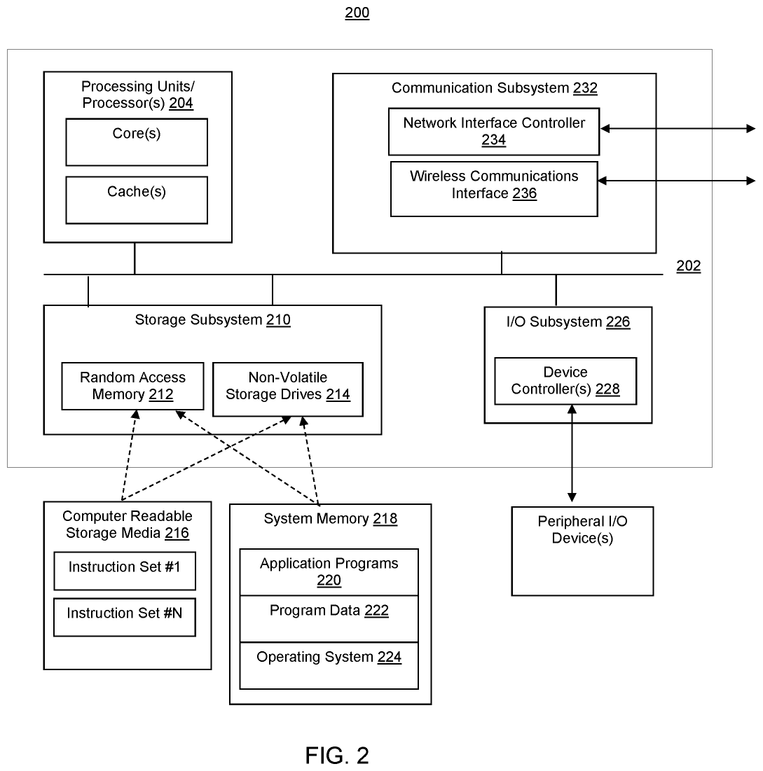

The embodiments shown in FIGS. 1-2 are thus one example of a distributed computing system and is not intended to be limiting. The subsystems and components within the server 102 and client devices 106 may be implemented in hardware, firmware, software, or combinations thereof. Various different subsystems and/or components 104 may be implemented on server 102. Users operating the client devices 106 may initiate one or more client applications to use services provided by these subsystems and components. Various different system configurations are possible in different distributed computing systems 100 and content distribution networks. Server 102 may be configured to run one or more server software applications or services, for example, web-based or cloud-based services, to support content distribution and interaction with client devices 106. Users operating client devices 106 may in turn utilize one or more client applications (e.g., virtual client applications) to interact with server 102 to utilize the services provided by these components. Client devices 106 may be configured to receive and execute client applications over one or more networks 120. Such client applications may be web browser based applications and/or standalone software applications, such as mobile device applications. Client devices 106 may receive client applications from server 102 or from other application providers (e.g., public or private application stores).

As shown in FIG. 1, various security and integration components 108 may be used to manage communications over network 120 (e.g., a file-based integration scheme or a service-based integration scheme). Security and integration components 108 may implement various security features for data transmission and storage, such as authenticating users or restricting access to unknown or unauthorized users,

As non-limiting examples, these security components 108 may comprise dedicated hardware, specialized networking components, and/or software (e.g., web servers, authentication servers, firewalls, routers, gateways, load balancers, etc.) within one or more data centers in one or more physical location and/or operated by one or more entities, and/or may be operated within a cloud infrastructure.

In various implementations, security and integration components 108 may transmit data between the various devices in the content distribution network 100. Security and integration components 108 also may use secure data transmission protocols and/or encryption (e.g., File Transfer Protocol (FTP), Secure File Transfer Protocol (SFTP), and/or Pretty Good Privacy (PGP) encryption) for data transfers, etc.).

In some embodiments, the security and integration components 108 may implement one or more web services (e.g., cross-domain and/or cross-platform web services) within the content distribution network 100, and may be developed for enterprise use in accordance with various web service standards (e.g., the Web Service Interoperability (WS-I) guidelines). For example, some web services may provide secure connections, authentication, and/or confidentiality throughout the network using technologies such as SSL, TLS, HTTP, HTTPS, WS-Security standard (providing secure SOAP messages using XML encryption), etc. In other examples, the security and integration components 108 may include specialized hardware, network appliances, and the like (e.g., hardware-accelerated SSL and HTTPS), possibly installed and configured between servers 102 and other network components, for providing secure web services, thereby allowing any external devices to communicate directly with the specialized hardware, network appliances, etc.

Computing environment 100 also may include one or more data stores 110, possibly including and/or residing on one or more back-end servers 112, operating in one or more data centers in one or more physical locations, and communicating with one or more other devices within one or more networks 120. In some cases, one or more data stores 110 may reside on a non-transitory storage medium within the server 102. In certain embodiments, data stores 110 and back-end servers 112 may reside in a storage-area network (SAN). Access to the data stores may be limited or denied based on the processes, user credentials, and/or devices attempting to interact with the data store.

With reference now to FIG. 2, a block diagram of an illustrative computer system is shown. The system 200 may correspond to any of the computing devices or servers of the network 100, or any other computing devices described herein. In this example, computer system 200 includes processing units 204 that communicate with a number of peripheral subsystems via a bus subsystem 202. These peripheral subsystems include, for example, a storage subsystem 210, an I/O subsystem 226, and a communications subsystem 232.

One or more processing units 204 may be implemented as one or more integrated circuits (e.g., a conventional micro-processor or microcontroller), and controls the operation of computer system 200. These processors may include single core and/or multicore (e.g., quad core, hexa-core, octo-core, ten-core, etc.) processors and processor caches. These processors 204 may execute a variety of resident software processes embodied in program code, and may maintain multiple concurrently executing programs or processes. Processor(s) 204 may also include one or more specialized processors, (e.g., digital signal processors (DSPs), outboard, graphics application-specific, and/or other processors).

Bus subsystem 202 provides a mechanism for intended communication between the various components and subsystems of computer system 200. Although bus subsystem 202 is shown schematically as a single bus, alternative embodiments of the bus subsystem may utilize multiple buses. Bus subsystem 202 may include a memory bus, memory controller, peripheral bus, and/or local bus using any of a variety of bus architectures (e.g. Industry Standard Architecture (ISA), Micro Channel Architecture (MCA), Enhanced ISA (EISA), Video Electronics Standards Association (VESA), and/or Peripheral Component Interconnect (PCI) bus, possibly implemented as a Mezzanine bus manufactured to the IEEE P1386.1 standard).

I/O subsystem 226 may include device controllers 228 for one or more user interface input devices and/or user interface output devices, possibly integrated with the computer system 200 (e.g., integrated audio/video systems, and/or touchscreen displays), or may be separate peripheral devices which are attachable/detachable from the computer system 200. Input may include keyboard or mouse input, audio input (e.g., spoken commands), motion sensing, gesture recognition (e.g., eye gestures), etc.

As non-limiting examples, input devices may include a keyboard, pointing devices (e.g., mouse, trackball, and associated input), touchpads, touch screens, scroll wheels, click wheels, dials, buttons, switches, keypad, audio input devices, voice command recognition systems, microphones, three dimensional (3D) mice, joysticks, pointing sticks, gamepads, graphic tablets, speakers, digital cameras, digital camcorders, portable media players, webcams, image scanners, fingerprint scanners, barcode readers, 3D scanners, 3D printers, laser rangefinders, eye gaze tracking devices, medical imaging input devices, MIDI keyboards, digital musical instruments, and the like.

In general, use of the term “output device” is intended to include all possible types of devices and mechanisms for outputting information from computer system 200 to a user or other computer. For example, output devices may include one or more display subsystems and/or display devices that visually convey text, graphics and audio/video information (e.g., cathode ray tube (CRT) displays, flat-panel devices, liquid crystal display (LCD) or plasma display devices, projection devices, touch screens, etc.), and/or non-visual displays such as audio output devices, etc. As non-limiting examples, output devices may include, indicator lights, monitors, printers, speakers, headphones, automotive navigation systems, plotters, voice output devices, modems, etc.

Computer system 200 may comprise one or more storage subsystems 210, comprising hardware and software components used for storing data and program instructions, such as system memory 218 and computer-readable storage media 216.

System memory 218 and/or computer-readable storage media 216 may store program instructions that are loadable and executable on processor(s) 204. For example, system memory 218 may load and execute an operating system 224, program data 222, server applications, client applications 220, Internet browsers, mid-tier applications, etc.

System memory 218 may further store data generated during execution of these instructions. System memory 218 may be stored in volatile memory (e.g., random access memory (RAM) 212, including static random access memory (SRAM) or dynamic random access memory (DRAM)). RAM 212 may contain data and/or program modules that are immediately accessible to and/or operated and executed by processing units 204.

System memory 218 may also be stored in non-volatile storage drives 214 (e.g., read-only memory (ROM), flash memory, etc.) For example, a basic input/output system (BIOS), containing the basic routines that help to transfer information between elements within computer system 200 (e.g., during start-up) may typically be stored in the non-volatile storage drives 214.

Storage subsystem 210 also may include one or more tangible computer-readable storage media 216 for storing the basic programming and data constructs that provide the functionality of some embodiments. For example, storage subsystem 210 may include software, programs, code modules, instructions, etc., that may be executed by a processor 204, in order to provide the functionality described herein. Data generated from the executed software, programs, code, modules, or instructions may be stored within a data storage repository within storage subsystem 210.

Storage subsystem 210 may also include a computer-readable storage media reader connected to computer-readable storage media 216. Computer-readable storage media 216 may contain program code, or portions of program code. Together and, optionally, in combination with system memory 218, computer-readable storage media 216 may comprehensively represent remote, local, fixed, and/or removable storage devices plus storage media for temporarily and/or more permanently containing, storing, transmitting, and retrieving computer-readable information.

Computer-readable storage media 216 may include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media. This can also include nontangible computer-readable media, such as data signals, data transmissions, or any other medium which can be used to transmit the desired information and which can be accessed by computer system 200.

By way of example, computer-readable storage media 216 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 216 may include, but is not limited to, Zip® drives, flash memory cards, universal serial bus (USB) flash drives, secure digital (SD) cards, DVD disks, digital video tape, and the like. Computer-readable storage media 216 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magneto-resistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 200.

Communications subsystem 232 may provide a communication interface from computer system 200 and external computing devices via one or more communication networks, including local area networks (LANs), wide area networks (WANs) (e.g., the Internet), and various wireless telecommunications networks. As illustrated in FIG. 2, the communications subsystem 232 may include, for example, one or more network interface controllers (NICs) 234, such as Ethernet cards, Asynchronous Transfer Mode NICs, Token Ring NICs, and the like, as well as one or more wireless communications interfaces 236, such as wireless network interface controllers (WNICs), wireless network adapters, and the like. Additionally and/or alternatively, the communications subsystem 232 may include one or more modems (telephone, satellite, cable, ISDN), synchronous or asynchronous digital subscriber line (DSL) units, Fire Wire® interfaces, USB® interfaces, and the like. Communications subsystem 236 also may include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof), global positioning system (GPS) receiver components, and/or other components.

In some embodiments, communications subsystem 232 may also receive input communication in the form of structured and/or unstructured data feeds, event streams, event updates, and the like, on behalf of one or more users who may use or access computer system 200. For example, communications subsystem 232 may be configured to receive data feeds in real-time from users of social networks and/or other communication services, web feeds such as Rich Site Summary (RSS) feeds, and/or real-time updates from one or more third party information sources (e.g., data aggregators). Additionally, communications subsystem 232 may be configured to receive data in the form of continuous data streams, which may include event streams of real-time events and/or event updates (e.g., sensor data applications, financial tickers, network performance measuring tools, clickstream analysis tools, automobile traffic monitoring, etc.). Communications subsystem 232 may output such structured and/or unstructured data feeds, event streams, event updates, and the like to one or more data stores that may be in communication with one or more streaming data source computers coupled to computer system 200.

The various physical components of the communications subsystem 232 may be detachable components coupled to the computer system 200 via a computer network, a FireWire® bus, or the like, and/or may be physically integrated onto a motherboard of the computer system 200. Communications subsystem 232 also may be implemented in whole or in part by software.

Due to the ever-changing nature of computers and networks, the description of computer system 200 depicted in the figure is intended only as a specific example. Many other configurations having more or fewer components than the system depicted in the figure are possible. For example, customized hardware might also be used and/or particular elements might be implemented in hardware, firmware, software, or a combination. Further, connection to other computing devices, such as network input/output devices, may be employed. Based on the disclosure and teachings provided herein, a person of ordinary skill in the art will appreciate other ways and/or methods to implement the various embodiments.

In the disclosed embodiments, the disclosed computing environment described above, executing one or more application programs 220 (including, and referred to, as a diagnostic probability calculator system or probability calculator system disclosed herein) may use aggregated data from screening tests or other diagnostic evaluation processes that incorporate known risk factors and assessment data, to objectively quantify a student's risk level for dyslexia or other conditions, described below. The probability calculator system may use this data and the results of the screening tests to execute calculations which provide a report including both a level of risk or probability of dyslexia for the subject or other user, and recommendations for an amount of intervention needed based on the level of risk. The calculator may be scaled up to more efficiently compute the risk factors for large groups of students.

The purpose of the probability calculator system is to calculate and determine how likely it is that an individual has dyslexia by accounting for and incorporating four factors: 1) a family history of dyslexia; 2) the prevalence rate of dyslexia in the population, 3) the results of one or more dyslexia screening tests (e.g., Pearson screening tests, selected by subjects or users, which include dyslexia screening tests); and 4) the psychometric properties of the screening test(s) (i.e., how accurately each screening test classifies students as having dyslexia or not).

The probability calculator system may make several calculations and determinations according to data stored in data store 110. As non-limiting examples, the data stored in data store 110 may include: prior probability data, which may further include a default value and/or a range of numbers representing the likelihood of dyslexia based on familial risk, or a default value or range of numbers representing an average prevalence rate of dyslexia; sensitivity or specificity of specific screening tests; false positives or false negatives of each test derived from the specificity or specificity of the tests; etc., as described in more detail below.

The data stored in data storage may be generated and aggregated by completing a review of existing literature, and/or by conducting and administering research studies related to the screening tests and the conditions that they screen for. The results of such a literature review and/or research studies may then be compiled and aggregated within data store 110. This data may be used by the diagnostic probability calculator system to develop models and/or prototypes used to determine the likelihood of the condition within each subject or other user as described below.

The probability calculator system may use the results of literature reviews or research studies of family history and/or the prevalence rate of dyslexia in the population to determine a prior probability factor, which is used as a factor in determining the likelihood of dyslexia for the subject or other user.

One or more studies may be relied on to determine prior probability of a subject or other user having dyslexia may include the user's history or the user's family history. As a non-limiting example, these studies may reveal that multiple factors contribute to the probability of dyslexia, including first, whether a subject had delays in speech and language development as a child, such as late onset of talking (speaking in single words) or delays in combining words. Another factor may include whether the subject has a first degree biological relative (sibling, parent) with a history of dyslexia. The results of the studies, or information found in various literature may further reveal ranges or means, for example that 29-66% of the children at familial risk have been found to develop dyslexia.

The disclosed system may update data store 110 to reflect the results of the studies. For example, given the 33-66% range of children at familial risk, the disclosed system may store 0.45, representing a 45% likelihood of dyslexia if the user has a family history, specifically a first degree relative that has dyslexia.

Another study, or other relevant literature that may be relied on to determine probability of a subject or other user having dyslexia may include an average prevalence rate of dyslexia in the population. As a non-limiting example, these studies, or related research literature may reveal that a mean prevalence within the population ranges from 0.04-0.20, or 4-20% of the population.

The disclosed system may update data store 110 to reflect the results of the studies. For example, given the 4-20% range of prevalence of dyslexia within the population, the disclosed system may store 0.10, representing a 10% likelihood of dyslexia given the prevalence in the population.

The probability calculator system may further use the results of one or more dyslexia screening tests, and the psychometric properties of each screening test (i.e., how accurately the test classifies subjects as having or not having dyslexia), in determining the likelihood of dyslexia for the subject or other user. The accuracy of the screening tests may be determined, and the tests themselves may include, attributes that are predefined around the sensitivity and specificity of the assessments.

When a clinical study is done, the research and results need to show that the screening tests are able to differentiate between people that have dyslexia and people that don't. The sensitivity and specificity values reflect this differentiation, and are determined by collecting two samples, which include matched controls: a group with dyslexia and a group without. Sensitivity and specificity for each screening test may be determined based on how each of those groups perform on the screening test. The sensitivity and specificity of each test may be related to, and used to determine false positives and false negatives for each of the screening tests.

The sensitivity and/or specificity data may be included as part of the clinical data, and may be published and reported in the technical manuals used by the organizations that provide the screening tests, for example. The values for sensitivity and specificity may be calculated and/or requested from the psychometrics, but are not necessarily specific to the probability calculator system or its utilized equations.

The sensitivity and specificity may be calculated according to the following equations: The sensitivity may calculated by adding the number of true positives to the number of false negatives, and dividing the number of true positives by this sum (i.e., sensitivity=number of true positives/number of true positives+number of false negatives). The specificity may be calculated by adding the number of true negatives to the number of false positives and dividing the number of true negatives by this sum (i.e., specificity=number of true negatives/number of true negatives+number of false positives).

The probability calculator system may update data store 110 to reflect the sensitivity, specificity, false positives and false negatives identified through the relevant literature or through the research studies. As non-limiting examples, the specificity, sensitivity, false positives, and false negatives for each of the following tests may include:

WIAT-III Dyslexia Index: K-1 (with 90% confidence interval): specificity (true negative)—0.81; false positive—0.19; false negative—0.17; sensitivity—0.83.

WIAT-III Dyslexia Index: 2-12+: specificity (true negative)—0.78; false positive—0.22; false negative—0.1; sensitivity (true positive)—0.9.

KTEA-3 Dyslexia Index: K-1: specificity (true negative)—0.85; sensitivity—0.95; false positive—0.15.

KTEA-3 Dyslexia Index: 2-12+: specificity (true negative)—0.74; sensitivity—0.94; false positive—0.26.

Shaywitz DyslexiaScreen Form 0 (Kindergarten): specificity (true negative)—0.71; sensitivity—0.73; false positive—0.29.

Shaywitz DyslexiaScreen Form 1 (Grade 1): specificity (true negative)—0.88; sensitivity—0.7; false positive—0.12.

Shaywitz DyslexiaScreen Form 2 (Grade 2): specificity (true negative)—0.75; sensitivity—1; false positive—0.25.

Shaywitz DyslexiaScreen Form 3 (Grade 3): specificity (true negative)—0.82; sensitivity—0.94; false positive—0.18.

WRAT5 Reading Composite: specificity (true negative)—0.79; sensitivity—0.81; false positive—0.21.

KTEA-3 BA-3: specificity (true negative)—0.85; sensitivity—0.9; false positive—0.15.

Once the disclosed system has stored the research data in data store 110 (e.g., prior probability data including family history data and prevalence data, sensitivity, specificity, false positive, and false negative data for each screening test, etc.), the disclosed system, possibly server(s) 102, 112, may generate one or more GUIs used to gather necessary user inputs from subjects or other users.

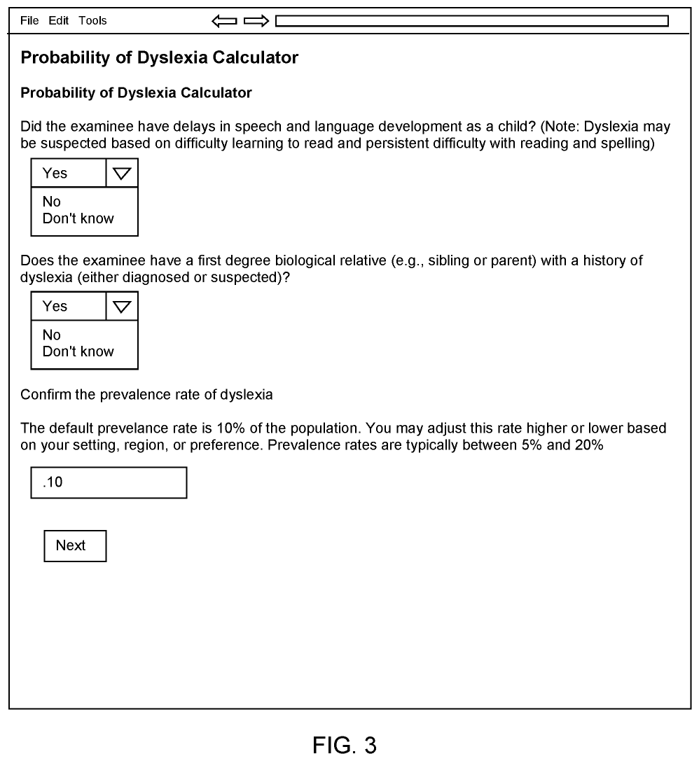

Turning now to FIG. 3, in some embodiments, a GUI may display a first request for user input (e.g., a first question) regarding whether the subject or other user has/had delays in speech and language development as a child. As seen in FIG. 3, the GUI may further include a first GUI control allowing a user to input a yes or no selection (or “Don't′ know”) in response to the first question.

The user device may receive the user's selection, and transmit the input response to the probability calculator system, possibly to server(s) 102, 112 for processing and possible storage in data store 110.

Returning to FIG. 3, the GUI may also include a second request for user input (e.g., a second question) regarding whether the subject has a family history of dyslexia. Specifically, the second request for user input may ask whether the subject or other user has or had a first degree biological relative, such as a sibling or parent, with a history of dyslexia. This history may include a diagnosed or suspected history of dyslexia.

As seen in FIG. 3, the GUI may further include a second GUI control allowing a user to input a yes or no selection (or “Don't know) in response to the second question. In some embodiments, the second request for user input and the second GUI control may be displayed only if the user selected “no” or “don't know” in response to the first GUI control. The client device 106 may receive the user's selection, and transmit the input response to the probability calculator system, possible server 102, 112, for processing and possible storage in data store 110.

Returning to FIG. 3, the GUI may also include a third request for user input, possibly including a confirmation of the default prevalence rate in the population, or a modification to the default value input by the user, explained in more detail below. Specifically, the third request for user input may request that the user either leave the automatically generated default prevalence rate (as stored in data store 110), or adjust the rate higher or lower, based on their setting, region, or preference, preferably between the rates of 5% and 20% in the non-limiting example embodiment in FIG. 3, but may be adjusted to any number (e.g., between 0.01. and 0.99).

As seen in FIG. 3, the GUI may further include a third GUI control automatically populated with the default prevalence value stored in data store 110, but may also able to accept input from the user to adjust the default value. In some embodiments, the third request for user input and the third GUI control may be displayed only if the user selected “no” or “don't know” from the first or second GUI controls in response to the first and/or the second question(s).

The client device 106 may receive the user's selection, and transmit the input response(s) to the probability calculator system, possibly server 102, 112 for processing and possible storage in data store 110.

The disclosed embodiments allow users to include the results from one or more dyslexia screening measures, such as teacher surveys and/or behavioral measures, in the probability calculation. Thus, the subject or other user may utilize additional dyslexia screening tests in order to refine the dyslexia probability calculations even further. In non-limiting example embodiments including two dyslexia screening tests, the use of two dyslexia screening tests, rather than a single dyslexia screening test, encourages a two-step dyslexia screening process—a best practice for a time efficient workflow that minimizes false positives.

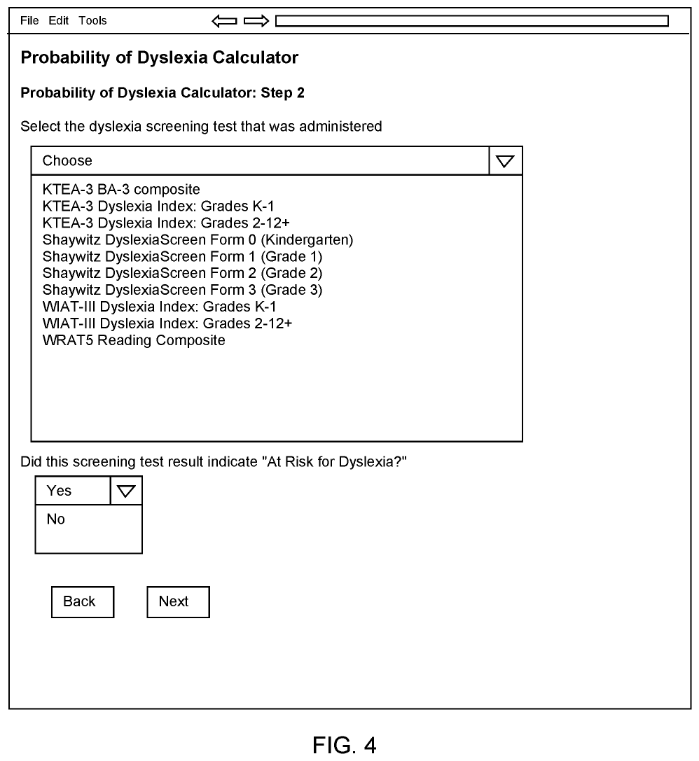

Turning now to FIG. 4, after submitting the user input in the first GUI (in this non-limiting example), the probability calculator system, possibly server(s) 102, 112 may generate and display, on the client device 106, a subsequent GUI. The GUI may include a first request for user input selecting, possibly from a dropdown list as in FIG. 4, a first dyslexia screening test administered to the subject or other user.

As seen in FIG. 4, the GUI may further include a first GUI control listing all of the available dyslexia screening tests within the disclosed system, allowing a user to select from the list a first dyslexia screening tests administered to the subject or other user. In embodiments in which multiple dyslexia screening tests were administered to the user, the user should select the dyslexia screening test administered first. As a non-limiting example, in FIG. 4, the subject or other user may select “Shaywitz DyslexiaScreen Form 1 (Grade 1).”

Returning to FIG. 4, the GUI may also include a second request for user input (e.g., a question) regarding whether the screening test result indicated “At Risk for Dyslexia.” As seen in FIG. 4, the GUI may further include a second GUI control allowing a user to input a yes or no selection in response to the question. The client device 106 may receive the user's selection, and transmit the input response to the probability calculator system, possibly server 102, 112 for processing and possible storage in data store 110.

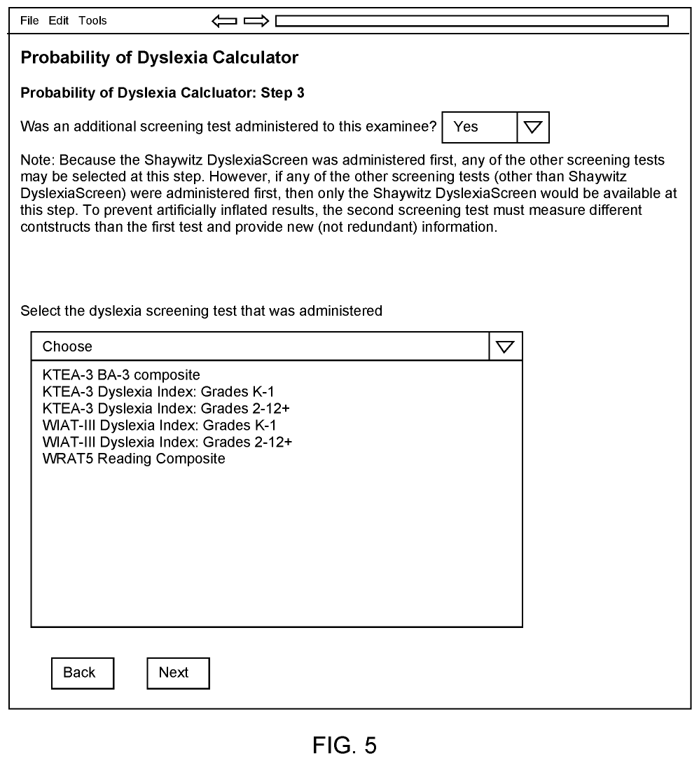

Turning now to FIG. 5, in embodiments that include two dyslexia screening tests, after submitting the user input in the second GUI (in this non-limiting example), the probability calculator system, possibly server(s) 102, 112 may generate and display, on the client device 106, another subsequent GUI. The GUI may include a first request for user input (e.g., a first question) regarding whether an additional screening test was administered to the subject/user. As seen in FIG. 5, the GUI may further include a first GUI control allowing a user to input a yes or no selection in response to the first question.

Returning now to FIG. 5, the GUI may include a second request for user input selecting, possibly from a dropdown list as in FIG. 5, a second dyslexia screening test administered to the subject or other user. As seen in FIG. 5, the GUI may further include a second GUI control listing all of the available dyslexia screening tests within the disclosed system, allowing a user to select from the list, a second dyslexia screening tests administered to the subject or other user. However, unlike the example dropdown GUI control in FIG. 4, the third GUI (in this example), would not include the option to select the previously-selected dyslexia screening test (“Shaywitz DyslexiaScreen Form 1 (Grade 1)” in the non-limiting example above) in order to give variation to the dyslexia probability calculations. As a non-limiting example, in FIG. 5, the subject or other user may select “KTEA-3 Dyslexia Index: Grades K-1.”

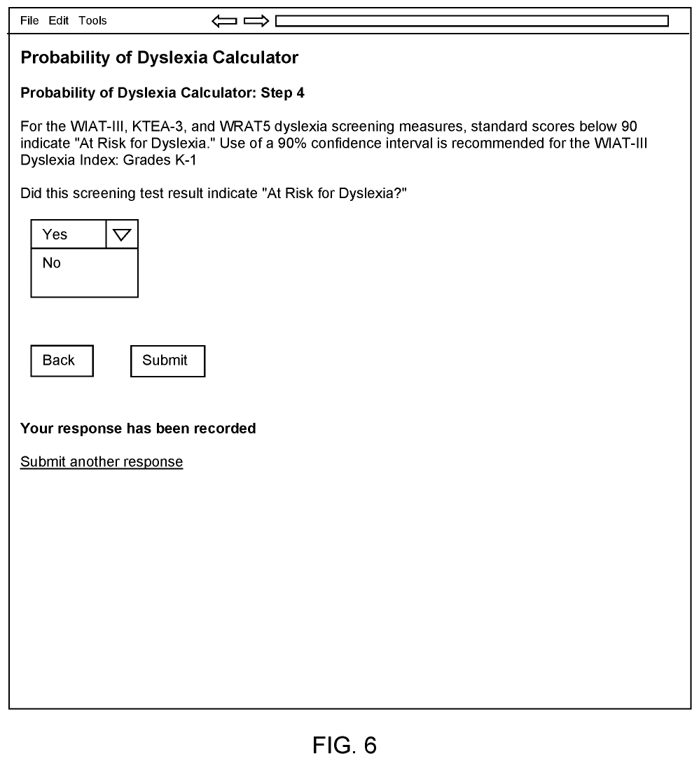

Turning now to FIG. 6, in embodiments that include two dyslexia screening tests, after submitting the user input in the third GUI (in this non-limiting example), the probability calculator system, possibly server(s) 102, 112 may generate and display, on the client device 106, another subsequent GUI. This GUI may also include a request for user input (e.g., a question) regarding whether the screening test result indicated “At Risk for Dyslexia.” As seen in FIG. 6, the GUI may further include a GUI control allowing a user to input a yes or no selection in response to the question. The client device 106 may receive the user's selection, and transmit the input response, as well as submit all data from the user inputs to the probability calculator system, possibly server(s) 102, 112 for processing and possible storage in data store 110.

The probability calculator system, possibly server(s) 102, 112 may receive the user inputs from the GUIs described above, and determine/calculate the probability and/or risk of dyslexia for the subject or other user that input the data.

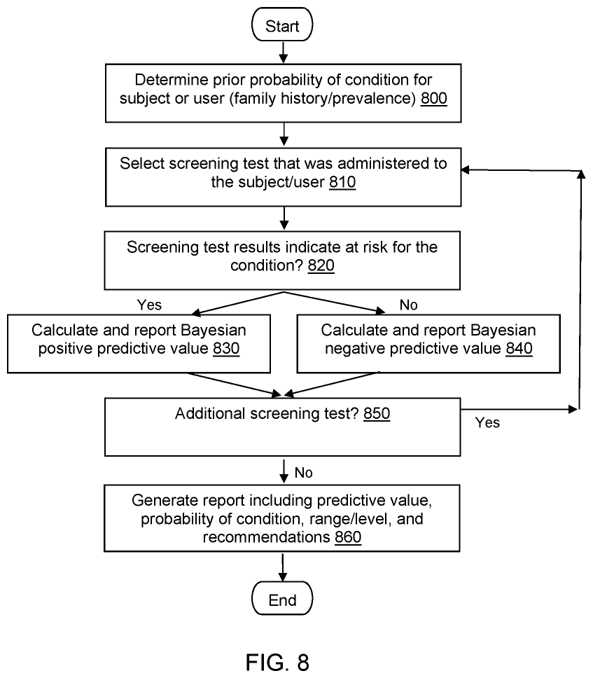

In embodiments in which a single test was administered, the disclosed system may determine/calculate the probability and/or risk of dyslexia using four steps, further reflected in Steps 800-860 of FIG. 8. These steps may include: 1) determining a prior probability of dyslexia; 2) selecting a dyslexia screening test that was administered; 3) determining whether the screening test result indicated “At Risk for Dyslexia;” and 4) reporting a Bayesian probability of dyslexia.

Step 1 above, also reflected in Step 800 of FIG. 8, calculates/determines a prior probability of dyslexia, and may be accomplished in two sub steps, not seen in FIG. 8. Step 1A includes determining whether the subject has had delays in speech and language development as a child, including late onset of talking (speaking in single words) or delays in combining words. Step 1B determines whether the subject has a first degree biological relative (sibling, parent) with a history of dyslexia. The disclosed system may access the data submitted from the GUIs above, and, determine if the subject has input a “yes” response to the question of whether they have had delays in speech and language development as a child, including late onset of talking (speaking in single words) or delays in combining words. In some embodiments, if the subject or other user input a “no” or “don't′ know” response, the disclosed system may proceed to step 1B, and access the data submitted from the GUIs above, to determine whether the subject has a first degree biological relative (sibling, parent) with a history of dyslexia.

The disclosed system may access the data submitted from the GUIs above, and, if the subject input a “yes” response, indicating that they have a first degree biological relative (sibling, parent) with a history of dyslexia, the disclosed system may access data store 110 to identify the default prior probability, and assign this value to the subject or other user in association with their probability of having dyslexia.

As a non-limiting example, as noted above, a default value (e.g., 0.45) may be stored in data store 110, based on the default value of prior probability according to the studies/literature. In the example above, the average likelihood of dyslexia based on family history was between 33 and 66%. Thus, an average likelihood of 45% was stored in the database, and a value of 0.45 may be assigned to the subject or other user in determining prior probability of dyslexia.

However, if the subject/user selected a “no” or “don't know” in response to the question of whether they have a first degree biological relative with a history of dyslexia, the disclosed system may determine probability of dyslexia using a third step, step 1C (not shown in FIG. 8), which determines the prior probability of dyslexia according to a prevalence rate of dyslexia in the population. In this step 1C, if the user input indicates that the subject does not have a family history of dyslexia, the disclosed system may calculate the prior probability of the subject/user having dyslexia according to either a default value for prevalence stored in data store 110 (based on the relevant literature or studies noted above), or may calculate the prior probability of the subject/user having dyslexia according to a value input by the user into the appropriate GUI described above.

For example, according to the example relevant literature and studies noted above, prevalence rates may be between 4 and 20%. Thus, data store 110 may have stored a default prevalence value of 0.10 or 10%. Therefore, if the subject/user has selected “no” or “don't′ know” regarding their family history of dyslexia, the disclosed system may access data store 110 and assign the subject/user a prior probability of 10%, based on the default value of the prevalence rate stored in data store 110.

However, if the subject/user selected the option to input a value adjusting the default prevalence value higher or lower based on their setting, region, or preference, the disclosed system may override the default value stored in data store 110, and assign the prevalence value for the subject/user to the prevalence rate they input in the GUIs described above. (e.g., 0.15).

Once the disclosed system has determined the prior probability of dyslexia for the subject/user, the disclosed system may continue to determine the probability of dyslexia by moving to step 2, also reflected in Step 810 of FIG. 8, identifying the selected dyslexia screening test that was administered. If the subject/user has taken more than one screening test (reflected in Step 850 of FIG. 8), this test will reflect the first screening test taken by the user. The disclosed system may identify this screening test by identifying the screening test selected by the subject/user in the GUIs described above.

Once the disclosed system has identified the (first) screening test selected by the user, the disclosed system may continue to determine the probability of dyslexia by moving to step 3, determining whether the (first) screening test result indicates that the subject/user is “At Risk for Dyslexia,” also reflected in Step 820 of FIG. 8. For example, for screening purposes, standard scores below 90 on the WIAT-III, KTEA-3, and WRAT5 measures indicate “At Risk for Dyslexia.” Use of a 90% confidence interval is recommended for the WIAT-III Dyslexia Index: Grades K-1.

The disclosed system may access the data submitted from the GUIs above, and, if the subject input a “yes” response, declaring that the screening test indicates that they are at risk for dyslexia, then the disclosed system may proceed to step 4, also reflected in Step 830 and 860 of FIG. 8, in which the disclosed system generates a GUI to report a Bayesian positive predictive value, as described in more detail below. However, if the subject input a “no” response, declaring that the screening test indicates that they are not at risk for dyslexia, then the disclosed system may proceed to step 4, also reflected in Steps 840 and 860 of FIG. 8, in which the disclosed system generates a GUI to report a Bayesian negative predictive value (1−the negative predictive value), as described in more detail below.

As noted above, the report generated in step 4, or Steps 830 and 840, includes a Bayesian positive predictive value and/or a Bayesian negative predictive value (1−negative predictive value). These values use a Bayes theorem probability calculation in order to estimate the likelihood of diagnostic conditions (in this case, dyslexia) given similar types of inputs, thereby quantifying a person's risk for the condition, such as dyslexia, which informs their diagnosis.

However, despite the disclosed embodiments using the Bayesian positive predictive value and/or Bayesian negative predictive value to determine the probability of dyslexia, the example of dyslexia is non-limiting, and could be used to estimate the likelihood of additional conditions given similar inputs in medical or psychological fields, including cancer, depression, etc., as non-limiting examples. The probability calculations or estimations would enable professionals in these fields to quantify a person's risk for a condition, thereby informing their diagnosis.

In some embodiments, the Bayesian positive predictive value for a selected and/or identified screening test, or the probability of dyslexia given a positive test result, may be calculated by multiplying the prior probability of dyslexia (“prior”) by the sensitivity, and dividing that result by the prior probability multiplied by the sensitivity plus 1 minus the prior probability times 1 minus the specificity, or in other words, prior*sensitivity/(prior*sensitivity+(1−prior)*(1−specificity).

The disclosed system may therefore calculate the Bayesian positive predictive value for a dyslexia screening test selected by a user in the GUIs described above, by calculating the prior probability as described in detail above, identifying, within data store 110, the sensitivity and specificity for the identified dyslexia screening test according to the relevant literature and results of studies as described above, and apply the formula above for calculating the Bayesian positive predictive value.

In some embodiments, the Bayesian negative predictive value for a selected and/or identified screening test, or the probability of dyslexia given a negative test result, may be calculated by multiplying 1 minus the prior probability by the sensitivity, and dividing that by 1 minus the sensitivity times the prior plus the specificity multiplied by 1 minus the prior probability, or in other words, (1−prior)*specificity/((1−sensitivity)*prior+specificity*(1−prior)).

The disclosed system may therefore calculate the Bayesian negative predictive value for a dyslexia screening test selected by a user in the GUIs described above, by calculating the prior probability as described in detail above, identifying, within data store 110, the sensitivity and specificity for the identified dyslexia screening test according to the relevant literature and results of studies as described above, and apply the formula above for calculating the Bayesian negative predictive value.

The calculation of the Bayesian probability of dyslexia incorporating any known risk factors is therefore based not only on the subject's prior probability of dyslexia and the subject's positive or negative results on the dyslexia screening test, but at a more granular level, is based on the dyslexia screening test's sensitivity and specificity, which are specific to each dyslexia screening test. The Bayesian positive and negative predictive values may, in turn, be used to determine false positives and false negatives in the results.

In the non-limiting example tests listed above, the formula for Bayesian positive predictive value and Bayesian negative predictive value would apply the example sensitivity and specificity values above as follows:

WIAT-III Dyslexia Index: K-1 (with 90% CI): the Bayesian positive predictive value, or the probability of dyslexia given a positive test result—prior*0.83/(prior*0.83+(1−prior)*(1−0.81)); the Bayesian negative predictive value, or the probability of dyslexia given a negative test result—(1−prior)*0.814(1−0.83)*prior+0.81*(1−prior)).

WIAT-III Dyslexia Index: 2-12+: the Bayesian positive predictive value, or the probability of dyslexia given a positive test result—prior*0.9/(prior*0.9+(1−prior)*(1−0.78)); the Bayesian negative predictive value, or the probability of dyslexia given a negative test result—(1−prior)*0.784(1−0.9)*prior+0.78*(1−prior)).

KTEA-3 Dyslexia Index: K-1: the Bayesian positive predictive value, or the probability of dyslexia given a positive test result—prior*0.95/(prior*0.95+(1−prior)*(1−0.85)); the Bayesian negative predictive value, or the probability of dyslexia given a negative test result—(1−prior)*0.854(1−0.95)*prior+0.85*(1−prior)).

KTEA-3 Dyslexia Index: 2-12+: the Bayesian positive predictive value, or the probability of dyslexia given a positive test result—prior*0.94/(prior*0.94+(1−prior)*(1−0.74)); the Bayesian negative predictive value, or the probability of dyslexia given a negative test result—(1−prior)*0.744(1−0.94)*prior+0.74*(1−prior)).

Shaywitz DyslexiaScreen Form 0 (Kindergarten): the Bayesian positive predictive value, or the probability of dyslexia given a positive test result—prior*0.73/(prior*0.73+(1−prior)*(1−0.71)); the Bayesian negative predictive value, or the probability of dyslexia given a negative test result—(1−prior)*0.714(1−0.73)*prior+0.71*(1−prior)).

Shaywitz DyslexiaScreen Form 1 (Grade 1): the Bayesian positive predictive value, or the probability of dyslexia given a positive test result—prior*0.7/(prior*0.7+(1−prior)*(1−0.88)); the Bayesian negative predictive value, or the probability of dyslexia given a negative test result—(1−prior)*0.884(1−0.7)*prior+0.88*(1−prior)).

Shaywitz DyslexiaScreen Form 2 (Grade 2): the Bayesian positive predictive value, or the probability of dyslexia given a positive test result—prior*1/(prior*1+(1−prior)*(1−0.75)); the Bayesian negative predictive value, or the probability of dyslexia given a negative test result—(1−prior)*0.754(1-1)*prior+0.75*(1−prior)).

Shaywitz DyslexiaScreen Form 3 (Grade 3): the Bayesian positive predictive value, or the probability of dyslexia given a positive test result—prior*0.94/(prior*0.94+(1−prior)*(1−0.82)); the Bayesian negative predictive value, or the probability of dyslexia given a negative test result—(1−prior)*0.824(1−0.94)*prior+0.82*(1−prior)).

WRAT5 Reading Composite: the Bayesian positive predictive value, or the probability of dyslexia given a positive test result—prior*0.81/(prior*0.81+(1−prior)*(1−0.79)); the Bayesian negative predictive value, or the probability of dyslexia given a negative test result—(1−prior)*0.794(1−0.81)*prior+0.79*(1−prior)).

KTEA-3 BA-3: the Bayesian positive predictive value, or the probability of dyslexia given a positive test result—prior*0.9/(prior*0.9+(1−prior)*(1−0.85)); the Bayesian negative predictive value, or the probability of dyslexia given a negative test result—(1−prior)*0.854(1−0.9)*prior+0.85*(1−prior)).

In some embodiments, a likelihood ratios method may be used. The disclosed embodiments may calculate likelihood ratios (LR), including positive likelihood ratios (PLR) and negative likelihood ratios (NLR). The likelihood ratios method may be calculated as follows:

For pretest odds, the likelihood ratios may be calculated by dividing the prior probability by 1 minus the prior, or prior/(1−prior).

For posttest odds, the likelihood ratios may be calculated by multiplying the pretest odds above by the likelihood ratios, or (pretest odds)*LR.

For posttest probability, the likelihood ratios may be calculated by dividing the posttest odds by the posttest odds+1, or (posttest odds)/(posttest odds+1).

For each of the tests described above, the PLR may be calculated by determining the sensitivity for that test, and dividing the sensitivity for that test by 1 minus the specificity, or PLR=sensitivity/(1−specificity). Any test with a PLR result greater than 5 (PLR=sensitivity/(1−specificity)>5), such as KTEA-3 Dyslexia Index: K-1, KTEA-3 BA-3, Shaywitz DyslexiaScreen Form 1 (Grade 1), or Shaywitz DyslexiaScreen Form 1 (Grade 1), as non-limiting examples, may be ruled in.

For each of these tests, the NLR may be calculated by determining the sensitivity for that test, and subtracting the sensitivity from one, then dividing that by the specificity, or NLR=(1−sensitivity)/specificity. Any test with a NLR result less than 0.2 (NLR=(1−sensitivity)/specificity<0.2), such as WIAT-III Dyslexia Index: 2-12+, KTEA-3 Dyslexia Index: K-1, KTEA-3 Dyslexia Index: 2-12+, Shaywitz DyslexiaScreen Form 2 (Grade 2), Shaywitz DyslexiaScreen Form 3 (Grade 3), and KTEA-3 BA-3, as non-limiting examples, may be ruled out.

Thus, the PLA and the NLA are as follows for the following non-limiting example tests: WIAT-III Dyslexia Index: K-1: PLR=4.368421, NLR=0.209877; WIAT-III Dyslexia Index: 2-12+: PLR=4.090909, NLR=0.128205; KTEA-3 Dyslexia Index: K-1: PLR=6.333333, NLR=0.058824; KTEA-3 Dyslexia Index: 2-12+: PLR=3.615385, NLR=0.081081; Shaywitz DyslexiaScreen Form 0 (Kindergarten): PLR=2.517241, NLR=0.380282; Shaywitz DyslexiaScreen Form 1 (Grade 1): PLR=5.833333, NLR=0.340909; Shaywitz DyslexiaScreen Form 2 (Grade 2): PLR=4, NLR=0; Shaywitz DyslexiaScreen Form 3 (Grade 3): PLR=5.222222, NLR=0.073171; WRAT5 Reading Composite: PLR=3.857143, NLR=0.240506; KTEA-3 BA-3: PLR=6, NLR=0.117647.

The disclosed system may use the data and calculations described above to determine a Bayesian positive predictive value and a Bayesian negative predictive value for the (first) dyslexia screening test selected from the GUIs described above. As non-limiting examples, the disclosed system may determine the following Bayesian positive predictive values and Bayesian negative predictive values for the following dyslexia screening tests disclosed above:

KTEA-3 BA-3: Bayesian positive predictive value—0.40, Bayesian negative predictive value—0.99; KTEA-3 Dyslexia Index: K-1: Bayesian positive predictive value—0.41, Bayesian negative predictive value—0.99; KTEA-3 Dyslexia Index: 2-12+: Bayesian positive predictive value—0.29, Bayesian negative predictive value—0.99; Shaywitz DyslexiaScreen Form 0 (Kindergarten): Bayesian positive predictive value—0.22, Bayesian negative predictive value—0.96; Shaywitz DyslexiaScreen Form 1 (Grade 1): Bayesian positive predictive value—0.39, Bayesian negative predictive value—0.96; Shaywitz DyslexiaScreen Form 2 (Grade 2): Bayesian positive predictive value—0.31, Bayesian negative predictive value—1.00; Shaywitz DyslexiaScreen Form 3 (Grade 3): Bayesian positive predictive value—0.37, Bayesian negative predictive value—0.99; WIAT-III Dyslexia Index: K-1: Bayesian positive predictive value—0.33, Bayesian negative predictive value—0.98; WIAT-III Dyslexia Index: 2-12+: Bayesian positive predictive value—0.31, Bayesian negative predictive value—0.99; WRAT5 Reading Composite: Bayesian positive predictive value—0.30, Bayesian negative predictive value—0.97.

Once the disclosed system has calculated the Bayesian probability of dyslexia based on the subject's prior probability and results from a first dyslexia screening test, the disclosed system may determine, based on the user input from the GUIs described above, whether an additional dyslexia screening test was administered to the subject, as seen in Step 850 of FIG. 8. If not, the report described below may include instructions to consider administering an additional dyslexia screening test as part of a two-step screening process to refine the calculated probability estimate.

However, if the disclosed system determines that an additional dyslexia screening test was administered to the subject/user, based on the input from the GUIs described above and as seen in Step 860 of FIG. 8, the disclosed system may proceed to steps 6-9, repeating steps 1-4, or Steps 810-850 in FIG. 8, for the additional dyslexia screening test, according to the description of steps 1-4 above.

Thus, in step 5, or Step 850 in FIG. 8, the disclosed system determines whether an additional dyslexia screening test was administered to this subject as part of the multi-step screening process. If so, the disclosed system proceeds to step 6 (810) and identifies the dyslexia screening test that was administered next. In step 7 (820), the disclosed system determines whether the results of the second screening test indicate “At Risk for Dyslexia.” If so, the disclosed system continues to step 9 (report revised Bayesian probability of dyslexia) and reports a positive predictive value for the identified dyslexia screening test (Step 830, 860). If not, the disclosed system continues to step 9 and reports a negative predictive value (1−negative predictive value) for the identified dyslexia screening test (Step 840, 860).

Repeating these steps starts the equation over in the context of the second test. These additional steps may provide the option of giving an additional screening test in order to refine the probability even further. The calculation is based on Bayesian probability reported in Step 4, the positive or negative test result reported in Step 7, and the test's sensitivity and false positive rate. Given the differences in the results of the dyslexia screening tests, the results of the second test, as compared to the first test, provide variance to the equation, thereby refining the prior probability for each equation calculated for each screening test.

The disclosed system may then combine the results from the first and second tests, which improves the predictive validity of tests to help administrators make better decisions for subjects or other users. As non-limiting examples, after analysis of the second dyslexia screening test from steps 6-9, the disclosed system may determine the following Bayesian positive predictive values and Bayesian negative predictive values for the following dyslexia screening tests disclosed above:

WIAT-III Dyslexia Index: K-1 (with 90% confidence interval): Bayesian positive predictive value—0.04, Bayesian negative predictive value—1.00; WIAT-III Dyslexia Index: 2-12+: Bayesian positive predictive value—0.04, Bayesian negative predictive value—1.00; KTEA-3 Dyslexia Index: K-1: Bayesian positive predictive value—0.06, Bayesian negative predictive value—1.00; KTEA-3 Dyslexia Index: 2-12+: Bayesian positive predictive value—0.04, Bayesian negative predictive value—1.00; Shaywitz DyslexiaScreen Form 0 (Kindergarten): Bayesian positive predictive value—0.02, Bayesian negative predictive value—1.00; Shaywitz DyslexiaScreen Form 1 (Grade 1): Bayesian positive predictive value—0.06, Bayesian negative predictive value—1.00; Shaywitz DyslexiaScreen Form 2 (Grade 2): Bayesian positive predictive value—0.04, Bayesian negative predictive value—1.00; Shaywitz DyslexiaScreen Form 3 (Grade 3): Bayesian positive predictive value—0.05, Bayesian negative predictive value—1.00; WRAT5 Reading Composite: Bayesian positive predictive value—0.04, Bayesian negative predictive value—1.00; KTEA-3 BA-3: Bayesian positive predictive value—0.06, Bayesian negative predictive value—1.00.

In some embodiments, a framework classifying the probability into ranges may be established for the results from the one or more dyslexia screening tests. These ranges may establish multiple levels, based on the probability (i.e., severity) of dyslexia which may, in turn, inform the intensity of the treatment evaluation response.

The resulting probabilities are interpreted categorically (low, medium, high) to provide a triage mechanism for tailoring recommendations to the degree of risk severity. This may allow practitioners to quickly determine which individuals are at the highest risk, or highest severity, for dyslexia, warranting an aggressive evaluation/treatment response.

As a non-limiting example, the framework may include levels for low, medium, and high risk. In this example, the 0-20% range may be considered low risk, the ˜21-74% range may be considered medium or moderate risk, and the ˜75-100% range may be considered high risk.

These ranges within the classification framework may be at the discretion of a system administrator based on their experience, who may determine the ranges and store them in data store 110 or as part of the logic of the disclosed system. As a non-limiting example, the system administrator may set the low range as 0-20% because many people think that dyslexia occurs in 20% of the population, and, although high by some estimates, would apply to almost all subjects generally. Similarly, the top 25% is a reasonable break to establish a high category.

The framework classifying the probability into ranges may include, or be associated in data store 110, with recommendations, which may help subjects at the highest risk get the best resources and the most intensive interventions sooner.

As a non-limiting example, recommendations for levels of high risk subjects (e.g., those in the ˜75-100% range of the framework) may include “A comprehensive evaluation and an intensive, individualized intervention approach is recommended as soon as possible,” representing a more aggressive response. These subjects may need a comprehensive evaluation, and the most aggressive intervention plan that is offered.

For those subjects at moderate risk (e.g., moderate risk levels), recommendations may include “More in-depth evaluation and/or evidence based intervention is recommended,” and for low risk subjects (e.g., low risk levels), recommendations may include “Consider alternative reasons for academic difficulty (if needed), and ensure high quality academic resources are available.

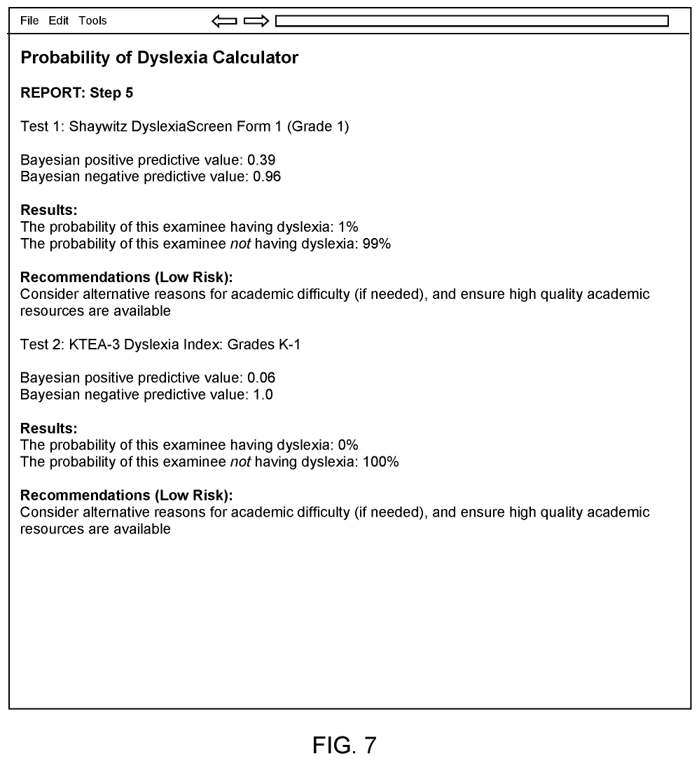

In steps 4 and 9 described above and reflected in FIG. 7 and step 860 of FIG. 8, the disclosed system may generate a report for each of the dyslexia screening tests administered to the subject/user. As non-limiting examples, such as that seen in FIG. 7, this report may include the Bayesian positive predictive value and the Bayesian negative predictive value for each of the dyslexia screening tests administered, as well as the results of each of the dyslexia screening tests.

For each of the dyslexia screening tests, the disclosed system may identify the level within the framework associated with the results, and may further identify the recommendation associated with the identified level. The disclosed system may then include the recommendation within the report. The disclosed system may then transmit the report through the network for display on the subject/user's device.

In some embodiments, the report may include a dial labeled “Probability of Dyslexia” (not shown in FIG. 7) similar to a speedometer on an automobile, which is divided according to the different levels within the framework. As non-limiting examples, such a dial may include a color code, so that green would be low risk, orange would be moderate risk, and red would be high risk.

Various use cases may demonstrate the utility of the disclosed embodiments. In a first use case, a special education teacher or speech language pathologist may use the probability calculator system as part of tier 1 (universal) or tier 2 (targeted) dyslexia screenings. Students with a high probability of dyslexia may be given a more comprehensive evaluation and the most intensive intervention right away. Students with moderate probability of dyslexia are given a tier 2 intervention and frequent progress monitoring. Students with low probability are monitored within tier 1. In a second example, a school psychologist may desire to use the probability calculator system as part of a tier 3 comprehensive evaluation to quantify family history and probability of diagnosis, and provides additional levels of justification for next steps.

As noted above, the disclosed embodiments may be used for any diagnosis of conditions, and are not limited to dyslexia diagnosis. The probability calculator system may also potentially be used for other conditions. For example, the probability calculator system may also be expanded and used to diagnose tests for ADHD, or any other diagnosis that are genetically linked. In this example, the family history, the prevalence, and the sensitivity and specificity measures ADHD may be substituted for a family history, prevalence and the sensitivity and specificity measures of dyslexia in the non-limiting examples, producing a similar result.

These example embodiments are non-limiting. The probability calculator system may easily be applied to other diagnoses in addition to dyslexia, and to additional products in other areas/markets.

Similarly, the disclosed example dyslexia screening tests and/or other products used in the disclosed examples are non-limiting. The disclosed embodiments could be generic to the subject matter (e.g., a person's political affiliation) as long as the tests have sufficient specificity and knowledge of other values and/or measures. The disclosed embodiments may further be used as a decision support tool in certain platforms, or a probability report option that could be a plugin to existing products.

In summary, and as shown in FIG. 8, the disclosed system may include a system, comprising a server, including a computing device coupled to a network and including at least one processor executing instructions within a memory coupled to the server.

When the instructions are executed, the system may be configured to receive, from a GUI displayed on a user device, user input from a user operating the user device comprising: a determination of whether a prior probability of dyslexia exists for a user operating the user device (Step 800); a first selection of a dyslexia screening test administered to the user (Step 810); and a second selection indicating whether the dyslexia screening test indicated a risk of dyslexia (Step 820).

The instructions may then cause the system to: calculate a Bayesian positive predictive value responsive to a determination that the dyslexia screening test indicated a risk of dyslexia (Step 830); calculate a Bayesian negative predictive value responsive to a determination that the dyslexia screening test did not indicate a risk of dyslexia (Step 840); and generate a report GUI including: the Bayesian positive predictive value or the Bayesian negative predictive value; a probability of the user having dyslexia; and a recommendation, according to the probability of the user having dyslexia, representing an intensity of a treatment evaluation response (Step 860).

Other embodiments and uses of the above inventions will be apparent to those having ordinary skill in the art upon consideration of the specification and practice of the invention disclosed herein. The specification and examples given should be considered exemplary only, and it is contemplated that the appended claims will cover any other such embodiments or modifications as fall within the true scope of the invention.

The Abstract accompanying this specification is provided to enable the United States Patent and Trademark Office and the public generally to determine quickly from a cursory inspection the nature and gist of the technical disclosure and in no way intended for defining, determining, or limiting the present invention or any of its embodiments.

The invention claimed is:

1. A system, comprising:
a data store coupled to a network and storing, in association, a dyslexia screening test, and a sensitivity and a specificity for the dyslexia screening test;
a client device coupled to the network and comprising a Graphical User Interface (GUI) including:
a first GUI control receiving from a user operating the client device a first user input indicating whether a prior probability of dyslexia exists for the user,
a second GUI control receiving from the user a second user input selecting the dyslexia screening test administered to the user, and
a third GUI control receiving from the user a third user input indicating whether the dyslexia screening test indicated a risk of dyslexia;
a server, including a computing device coupled to the network and including at least one processor executing instructions within a memory coupled to the server which, when executed, cause the system to:
receive, from the client device, the first user input, the second user input, and the third user input;
calculate a Bayesian positive predictive value based on the sensitivity and specificity for the screening test, responsive to a determination that the dyslexia screening test indicated a risk of dyslexia;
calculate a Bayesian negative predictive value based on the sensitivity and specificity for the screening test, responsive to a determination that the dyslexia screening test did not indicate a risk of dyslexia;
generate a report GUI, for display on the client device including:
the Bayesian positive predictive value or the Bayesian negative predictive value;
a probability of the user having dyslexia; and
a recommendation, according to the probability of the user having dyslexia, representing an intensity of a treatment evaluation response.

2. The system of claim 1, wherein the prior probability of dyslexia is identified by:
the user having delays in speech and language development as a child; or
the user having a first degree biological relative with a history of dyslexia.

3. The system of claim 2, wherein, responsive to the user indicating, via a fourth GUI control displayed on the GUI, that the user did not have delays in speech and language development as a child, the GUI displays a fifth GUI control determining whether the user had a first degree biological relative with a history of dyslexia.

4. The system of claim 1, wherein the prior probability of dyslexia is identified by a prevalence rate of dyslexia in a population.

5. The system of claim 4, wherein the GUI displays a GUI control requesting a confirmation of a default prevalence rate displayed on the GUI.

6. A system, comprising a server, including a computing device coupled to a network and including at least one processor executing instructions within a memory coupled to the server which, when executed, cause the system to:
receive, from a Graphical User Interface (GUI) displayed on a user device, user input from a user operating the user device comprising:
a determination of whether a prior probability of dyslexia exists for a user operating the user device;
a first selection of a dyslexia screening test administered to the user; and
a second selection indicating whether the dyslexia screening test indicated a risk of dyslexia;
calculate a Bayesian positive predictive value responsive to a determination that the dyslexia screening test indicated a risk of dyslexia;
calculate a Bayesian negative predictive value responsive to a determination that the dyslexia screening test did not indicate a risk of dyslexia;
generate a report GUI including:
the Bayesian positive predictive value or the Bayesian negative predictive value;
a probability of the user having dyslexia; and
a recommendation, according to the probability of the user having dyslexia, representing an intensity of a treatment evaluation response.

7. The system of claim 6, wherein:
the user input received from the GUI further includes:
a third selection of a second dyslexia screening test administered to the user; and
a fourth selection indicating whether the second dyslexia screening test indicated a risk of dyslexia;
the instructions further cause the system to:
calculate a second Bayesian positive predictive value responsive to a determination that the second dyslexia screening test indicated a risk of dyslexia; and
calculate a second Bayesian negative predictive value responsive to a determination that the second dyslexia screening test did not indicate a risk of dyslexia.

8. The system of claim 6, wherein the Bayesian positive predictive value is calculated according to the prior probability, a sensitivity associated, in a data store coupled to the network, with the dyslexia screening test, and a specificity associated with the dyslexia screening test.

9. The system of claim 6, wherein the Bayesian negative predictive value is calculated according to the prior probability, a sensitivity associated, in a data store coupled to the network, with the dyslexia screening test, and a specificity associated with the dyslexia screening test.

10. The system of claim 6, wherein the Bayesian negative predictive value is calculated by subtracting the negative predictive value from 1.

11. The system of claim 6, wherein the recommendation is selected from a framework defining a plurality of levels associated, in a data store coupled to the network, with the probability of the user having dyslexia.

12. A method, comprising the steps of:
receiving, by a server including a computing device coupled to a network and including at least one processor executing instructions within a memory, from a Graphical User Interface (GUI) displayed on a user device, user input from a user operating the user device comprising:
a determination of whether a prior probability of dyslexia exists for a user operating the user device;
a first selection of a dyslexia screening test administered to the user; and
a second selection indicating whether the dyslexia screening test indicated a risk of dyslexia;
calculating, by the server, a Bayesian positive predictive value responsive to a determination that the dyslexia screening test indicated a risk of dyslexia;
calculating, by the server, a Bayesian negative predictive value responsive to a determination that the dyslexia screening test did not indicate a risk of dyslexia;
generating, by the server a report GUI including:
the Bayesian positive predictive value or the Bayesian negative predictive value;
a probability of the user having dyslexia; and
a recommendation, according to the probability of the user having dyslexia, representing an intensity of a treatment evaluation response.

13. The method of claim 12, wherein:
the user input received from the GUI further includes:
a third selection of a second dyslexia screening test administered to the user; and
a fourth selection indicating whether the second dyslexia screening test indicated a risk of dyslexia;
the method further comprises the steps of:
calculating, by the server, a second Bayesian positive predictive value responsive to a determination that the second dyslexia screening test indicated a risk of dyslexia; and
calculating, by the server, a second Bayesian negative predictive value responsive to a determination that the second dyslexia screening test did not indicate a risk of dyslexia.

14. The method of claim 12, wherein the prior probability of dyslexia is identified by:
the user having delays in speech and language development as a child; or
the user having a first degree biological relative with a history of dyslexia.

15. The method of claim 14, wherein, responsive to the user indicating, via a fourth GUI control displayed on the GUI, that the user did not have delays in speech and language development as a child, the GUI displays a fifth GUI control determining whether the user had a first degree biological relative with a history of dyslexia.

16. The method of claim 1, wherein:
the prior probability of dyslexia is identified by a prevalence rate of dyslexia in a population; and
the GUI displays a GUI control requesting a confirmation of a default prevalence rate displayed on the GUI.

17. The method of claim 12, wherein the Bayesian positive predictive value is calculated according to the prior probability, a sensitivity associated, in a data store coupled to the network, with the dyslexia screening test, and a specificity associated with the dyslexia screening test.

18. The method of claim 12, wherein the Bayesian negative predictive value is calculated according to the prior probability, a sensitivity associated, in a data store coupled to the network, with the dyslexia screening test, and a specificity associated with the dyslexia screening test.

19. The method of claim 12, wherein the Bayesian negative predictive value is calculated by subtracting the negative predictive value from 1.

20. The method of claim 12, wherein the recommendation is selected from a framework defining a plurality of levels associated, in a data store coupled to the network, with the probability of the user having dyslexia.
